(19) United States
(12) Patent Application Publication
(10) Pub. No.: US 2018/0330820 A1
(43) Pub. Date: Nov. 15, 2018

(54) CONTENT-DRIVEN PROBLEM LIST RANKING IN ELECTRONIC MEDICAL RECORDS

(71) Applicant: KONINKLIJKE PHILIPS N.V., EINDHOVEN (NL)

(72) Inventors: Merlijn Sevenster, Haarlem (NL); Paul Joseph Chang, Chicago, IL (US); Yuechen Qian, Lexington, MA (US)

(73) Assignee: Koninklijke Philips N.V., Eindhoven (NL)

(21) Appl. No.: 15/776,822

(22) PCT Filed: Nov. 25, 2016
(86) PCT No.: PCT/EP2016/078851

§ 371 Date: May 17, 2018

Related U.S. Application Data
(60) Provisional application No. 62/259,903, filed on Nov. 25, 2015.

(51) Int. Cl. G16H 50/20; G06F 17/30

(52) U.S. Cl. CPC G16H 50/20; G06F 17/3053

(57) ABSTRACT

A system and method perform the steps of retrieving a problem list with active diagnostic information items for a patient; constructing a clinical context for a current imaging exam based on retrieved relevant diagnostic information for the current imaging exam; determining a ranking scheme with relevance rules based on the clinical context, wherein the relevance rules rank a relevance of problem list diagnostic information items based on the clinical context for the current imaging exam; selecting a ranking scheme; and implementing the selected ranking scheme to sort the problem list diagnostic information items.

### BACKGROUND

The “problem list” is a field within the Electronic Medical Record (EMR), which contains active diagnosis information items for a patient. The problem list may be encoded as a list of items from the International Classification of Diseases (ICD) scheme. Medical professionals maintain the problem list to provide a current overview of the disease status for a patient, so that medical professionals may easily synthesize an understanding of the patient's medical history.

The clinical history section of medical documents for imaging exams usually includes information relevant to the medical professional for the current exam. Exemplary medical documents may include, for example, radiology and echo reports. An up-to-date and complete problem list will include all diagnostic information items from the medical exam report clinical history section.

Existing sorting methods generally sort problem lists by reverse chronological order, where the most recent diagnostic item is placed at the top of the problem list. For example, the most recent diagnostic item of “falls frequently” may be irrelevant to a radiologist for a current imaging exam, while the less recent diagnostic item “Diabetes mellitus” may be very relevant. Lengthy problem lists sorted in reverse chronological order are difficult for busy medical professionals to process, which may cause the medical professional to overlook critical diagnostic information entered further in the past, for example, chronic conditions.

### SUMMARY

A method, comprising: retrieving a problem list with active diagnostic information items for a patient; constructing a clinical context for a current imaging exam based on retrieved relevant diagnostic information for the current imaging exam; determining a ranking scheme with relevance rules based on the clinical context, wherein the relevance rules rank a relevance of the diagnostic information items of the problem list based on the clinical context for the current imaging exam; selecting a ranking scheme; and implementing the selected ranking scheme to sort the diagnostic information items of the problem list.

A system, comprising: a non-transitory computer readable storage medium storing an executable program; and a processor executing the executable program to cause the processor to: retrieve a problem list with active diagnostic information items for a patient; construct a clinical context for a current imaging exam based on retrieved relevant diagnostic information for the current imaging exam; determine a ranking scheme with relevance rules based on the clinical context, wherein the relevance rules rank a relevance of problem list diagnostic information items based on the clinical context for the current imaging exam; select a ranking scheme; and implement the selected ranking scheme to sort the problem list diagnostic information items.

A non-transitory computer-readable storage medium including a set of instructions executable by a processor, the set of instructions, when executed by the processor, causing the processor to perform operations, comprising: retrieving a problem list with active diagnostic information items for a patient; constructing a clinical context for a current imaging exam based on retrieved relevant diagnostic information for the current imaging exam; determining a ranking scheme with relevance rules based on the clinical context, wherein the relevance rules rank a relevance of problem list diagnostic information items based on the clinical context for the current imaging exam; selecting a ranking scheme; and implementing the selected ranking scheme to sort the problem list diagnostic information items.

### BRIEF DESCRIPTION OF THE DRAWINGS

FIG. 1 shows a schematic drawing of a system according to an exemplary embodiment.

FIG. 2 shows a flow diagram of a method according to a first exemplary embodiment.

FIG. 3 shows a problem list ranked by relevance, according to a first exemplary embodiment.

FIG. 4 shows a problem list ranked by reverse chronology, according to a first exemplary embodiment.

### DETAILED DESCRIPTION

The exemplary embodiments may be further understood with reference to the following description and the appended drawings, wherein like elements are referred to with the same reference numerals. The exemplary embodiments relate to systems and methods for ranking patient diagnostic information items in a problem list based on the content of the diagnostic information. Those skilled in the art will understand that users may include any type of medical professional, including, for example, doctors, nurses, medical technicians, etc. Although exemplary embodiments specifically describe ranking patient diagnostic information for medical documents, it will be understood by those of skill in the art that the systems and methods of the present disclosure may be used to rank diagnostic information for any type of study or exam within any of a variety of hospital settings.

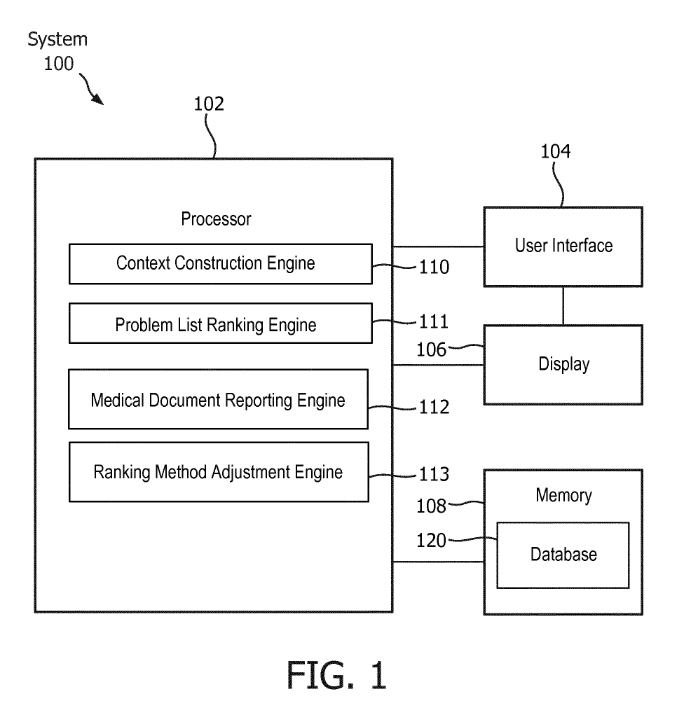

FIG. 1 shows an exemplary system 100 for ranking patient diagnostic information items within a problem list based on the content of the diagnostic information. The system 100 comprises a processor 102, a user interface 104, a display 106, and a memory 108. The memory 108 includes a database 120, which stores medical documents on imaging exams, including for example, radiology and cardiology echo reports for a patient. The database 120 includes for example an electronic medical record (EMR) and a Picture Archiving and Communications System (PACS) for storing problem lists of patient diagnostic items maintained by medical professional users. Imaging exams may include exams performed on MRI, CT, CR, ultrasound, etc., and problem lists may include diagnostic information items from the clinical history sections of reports for imaging exams. Those of skill in the art will understand that the method of the present disclosure may be used to rank diagnostic information items for any type of study or exam within any of a variety of hospital settings. The retrieved diagnostic information items may be viewed in a display 106, and ranked and reviewed via a user interface 104, e.g. an EMR interface. An exemplary embodiment of the user interface 104 may be an EMR interface with an Application Programming Interface (API).

The processor 102 includes a context construction engine 110, a problem list ranking engine 111, a medical document reporting engine 112, and a ranking method adjustment engine 113. Each of the engines will be described in greater detail below.

Those skilled in the art will understand that the engines 110-113 may be implemented by the processor 102 as, for example, lines of code that are executed by the processor 102, as firmware executed by the processor 102, as a function of the processor 102 being an application specific integrated circuit (ASIC), etc. By making selections on the user interface 104, the user may retrieve a problem list with diagnostic information items for a patient based on a patient identification number, for example, a medical record number (MRN), from the database 120, e.g. the electronic medical record (EMR). The context construction engine 110 retrieves relevant diagnostic information for the current imaging exam from the database 120, constructs a clinical context for the current imaging exam using the relevant diagnostic information, and makes the clinical context available for use by other applications. Diagnostic information relevant for a current imaging exam may include, for example, mode of image acquisition and imaged anatomy for a current imaging exam.

The problem list ranking engine 111 determines a ranking scheme with relevance rules based on the constructed clinical context, for sorting the diagnostic information items in the problem list. The ranking scheme relevance rules may be based on relevance themes or the hierarchical tree structure of the International Classification of Diseases (ICD). Using a selected ranking scheme, the problem list ranking engine 111 sorts the problem list depicted on the display 106. In an exemplary embodiment, the user may change the display of the problem list on display 106, by selecting an exemplary problem list ranking scheme of relevance or chronology, etc.

In an exemplary embodiment, the problem list ranking engine 111 may display the problem list as an advanced problem list display on the display 106, where the problem list items may be selected by users, using the user interface 104.

In another exemplary embodiment, the medical document reporting engine 112 may, using an exemplary template, convert the selected problem list items from the advanced problem list display on display 106, into natural language statements. The medical document reporting engine 112 subsequently makes the natural language statements accessible for user applications, for example, permitting access through the medical report editor application programming interface (API) or with an intermediate storage entity, e.g. Clipboard.

In another exemplary embodiment, the ranking method adjustment engine 113 may incorporate the manual user selections of problem list items on the advanced problem list display depicted on display 106 to refine or reinforce the relevance rules for the created ranking scheme used in the problem list ranking engine 111. For example, ranking method adjustment engine 113 may apply exception rules modeling manual adjustments, to refine the relevance rules used in the ranking scheme for the problem list ranking engine 111. In another example, the ranking method adjustment engine 113 may refine the relevance values in a contextually determined look-up table of the ranking scheme, by requesting user feedback in a dialogue, where the dialogue employs the hierarchy of the International Classification of Diseases (ICD), requesting feedback on relevance and contextual exam parameters, e.g. medical professional credentials, professional seniority, relevant exam anatomy, etc.

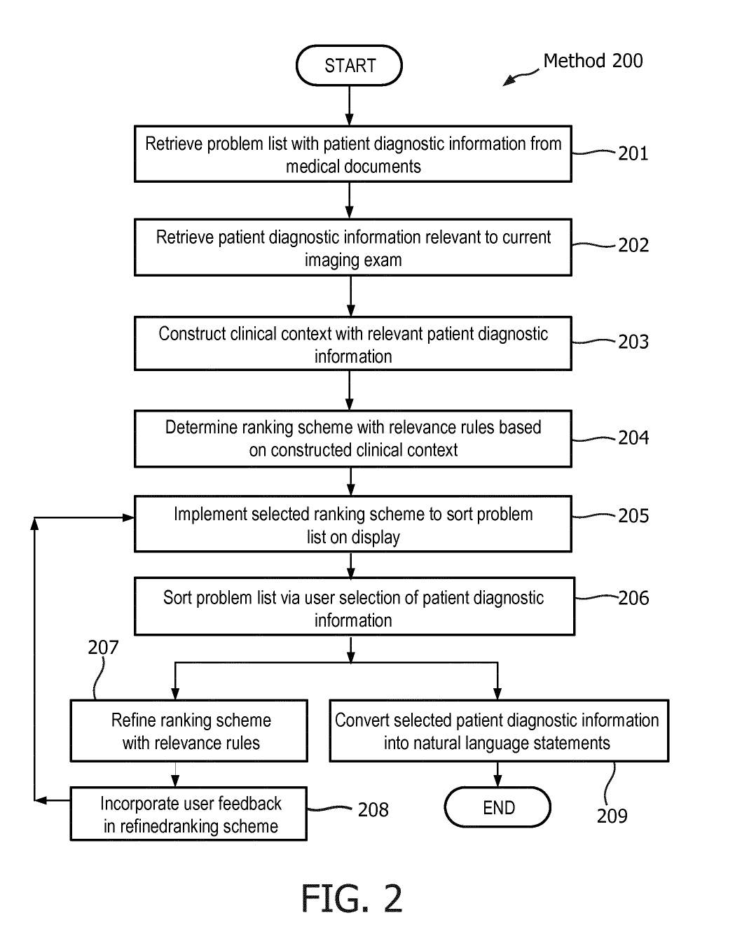

FIG. 2 shows a method 200 for ranking active patient diagnostic information items in a problem list based on the content of the diagnostic information, using the system 100 above. The method 200 comprises steps for retrieving a problem list and retrieving relevant patient diagnostic information, constructing a clinical context with the relevant patient diagnostic information, determining a ranking scheme based on the constructed clinical context, applying a ranking scheme to sort the displayed problem list, and displaying the sorted problem list, on a problem list display.

In step 201, the context construction engine 110 retrieves a problem list with diagnostic information items, based on a user selection of a patient by selecting a patient identification number, e.g. a medical reference number (MRN), on user interface 104, for example, from the database 120. The problem list is located in the database 120, e.g. the electronic medical record (EMR), and includes patient diagnostic information items from patient medical exams, including the clinical history section, and is maintained by medical professionals to depict a current overview of the patient disease status.

In step 202, the context construction engine 110 retrieves relevant patient diagnostic information for the current imaging exam using a patient identification number. In step 203, the context construction engine 110 constructs a clinical context for the current imaging exam using the relevant diagnostic information, and makes the clinical context available for use by other applications. The database 120 may comprise, for example, an electronic medical record (EMR) and the picture archiving and communication system (PACS) for storing problem lists of patient diagnostic information items maintained by medical professional users. Diagnostic information relevant for a current imaging exam may include, for example, mode of image acquisition (e.g. transthoracic, trans-esophageal, etc.), modality for image acquisition (e.g. computerized tomography “CT,” magnetic resonance “MR,” etc.), and imaged anatomy for the exam (e.g. brain). The retrieved relevant information may be normalized with reference to a protected vocabulary. For example, the retrieved information may have been processed with a concept extraction engine, which has also retrieved concepts from a medical ontology, e.g. SNOMED CT or RadLex. Each retrieved concept has a unique identifier, and concepts in the ontology are inter-related, e.g. through concept relationships of “is a” or a “part of.” With the inter-related ontology concepts, hierarchical reasoning may be applied for the retrieved concepts and retrieved information.

In step 204, the problem list ranking engine 111 determines a ranking scheme with relevance rules based on the constructed clinical context. In step 204, the problem list ranking engine 111 may determine a ranking scheme by modeling one or more ranking schemes, where each scheme models a distinct relevance theme, for example, relevance based on medical domain specialty (e.g. radiology); a user profile (e.g. resident, fellow, or attending physician); a user (e.g. Dr. A. Smith); and an exam date, etc. In an exemplary embodiment, relevance may be modeled in intervals. For example, the interval [0, 1] may indicate that an entry with relevance values between 0 and 1 has low relevance, and the interval [3, 5] may indicate that an entry with relevance values between 3 and 5 has high relevance. In another exemplary embodiment, relevance may be modeled as values from a controlled list, e.g. establishing numerical values for each of the low, medium, and high relevance levels. For example, a low relevance value may be 0.1, a medium relevance value may be 0.4, and a high relevance value may be 0.8.

In another exemplary embodiment of step 204, a ranking scheme may be determined as a set of relevance rules that employ the hierarchical tree structure of the International Classification of Diseases (ICD). An exemplary relevance rule may be: when the ICD code is subordinate to ICD node “malignant neoplasm” in the ICD hierarchy, the relevance is 0.8. The relevance rules may also use the constructed clinical context, for example, according to the following example: for the ICD code subordinate to the ICD node “head, face, and neck” in the ICD hierarchy, with the exam anatomy of “brain,” the relevance is 0.5. As another example, the relevance rules might apply the date of entry of the ICD code according to the following exemplary rule: when the ICD code is subordinate to “symptoms, signs, and ill-defined conditions” in the ICD hierarchy, and the ICD code was not entered in the last month, the relevance is 0.1. In another exemplary embodiment, a look-up table may apply contextual exam parameters in the constructed clinical context to map ICD codes to relevance values, so that users may look up relevance values based on specific ICD codes. Exemplary contextual exam parameters may include medical professional credentials, professional seniority, relevant exam anatomy, etc.

In step 205, the problem list ranking engine 111 implements a selected ranking scheme with relevance rules, to sort the problem list. Using the selected ranking scheme, the problem list ranking engine 111 sorts the problem list in the display 106. In an exemplary embodiment, the display 106 may be integrated with patient information. In another exemplary embodiment, the user may change the displayed ranking of the problem list by selecting an exemplary ranking scheme of relevance or chronology, etc.

In step 206, the problem list ranking engine 111 may display the problem list as a further sorted, advanced problem list display on the display 106, upon user selection of the user-selectable problem list items, e.g. the ICD codes, using the user interface 104. For example, a check box may precede each ICD code, or selected ICD codes may be highlighted or pre-selected on the user interface 104, so that the user may select ICD codes using check boxes, or the user may remove the selections of pre-selected ICD codes to tailor the selections of ICD codes. In another exemplary embodiment, highly relevant ICD codes may be pre-selected by default, based on the selected ranking scheme, and the user may remove the selections of pre-selected ICD codes. Upon user selection of the problem list items on user interface 104, the problem list ranking engine 111 may sort the problem list according to the user selections.

In step 209, the medical document reporting engine 112 may, using an exemplary template, convert the selected problem list diagnostic information items from the advanced problem list display on display 106, into natural language statements. The selected problem list items may include ICD codes. An exemplary natural language statement converted from the selected problem list items of “diabetes mellitus, colon cancer, and congestive heart failure (CHF),” may be, for example, “known relevant diagnoses include diabetes mellitus, colon cancer, and congestive heart failure (CHF).” In another exemplary embodiment, the template may be externally configurable. In step 209, the medical document reporting engine 112 makes the natural language statements accessible for user applications, for example, through the medical report editor application programming interface (API) or with an intermediate storage entity, e.g. Clipboard.

In step 207, the ranking method adjustment engine 113 may refine or reinforce the relevance rules for the created ranking scheme used in the problem list ranking engine 111, for example, by incorporating the manual user selections of problem list items in step 206. For example, once relevance rules for the problem list ranking engine 111 are determined for the ranking scheme in step 204, ranking method adjustment engine 113 may add relevance exception rules modeling manual adjustments to the ranking scheme. For example, the problem list ranking engine 111 may determine that all ICD codes that are “symptoms” are irrelevant and therefore, have low relevance. In an exemplary embodiment, the ranking method adjustment engine 113 may enter exceptions to the relevance rules for the problem list ranking engine 111 that the user wishes to add. For example, the ranking method adjustment engine 113 may enter certain ICD exception codes to the relevance rules that indicate these specific ICD codes with “symptoms” are high relevance. These exception rules further refine the relevance rules in the ranking scheme applied by the problem list ranking engine 111.

In an exemplary embodiment, the system 100 may learn the relevance of problem list items, based on previous user selections of problem list items in step 206. This learned relevance may be used to refine the relevance rules in the ranking scheme applied by the problem list ranking engine 111.

In step 208, in an exemplary embodiment, the ranking method adjustment engine 113 may refine or reinforce the ranking scheme by adjusting relevance values in a contextually determined look-up table, by requesting user feedback in a dialogue employing the hierarchy of the International Classification of Diseases (ICD). Exemplary feedback may be requested on contextual exam parameters, e.g. medical professional credentials, professional seniority, relevant exam anatomy, etc. An exemplary dialogue with the user may include, for example, the following consecutive user prompts asking: “1) You indicated that ‘diabetes mellitus without mention of complications, Type II or unspecified type, not stated as uncontrolled’ is highly relevant in the context of a Brain CT exam. Is this highly relevant in any context, yes or no?” 2) Is ‘diabetes mellitus without mention of complication’ highly relevant in any context, yes or no? 3) Is ‘diabetes mellitus’ highly relevant in any context, yes or no?” The ranking method adjustment engine 113 incorporates this user feedback to refine the relevance values for the ICD code look-up table incorporating relevance values and contextual parameters in the constructed clinical context, where the ranking scheme is implemented as a look-up table.

In step 208, additional dialogue with the user may be performed, requesting additional user feedback to refine the relevance values in a contextually determined ICD code look-up table, where the ranking scheme is implemented as a look-up table incorporating relevance values and contextual parameters in the constructed clinical context.

In an exemplary embodiment of step 207, in an off-line machine-learning method, the ranking method adjustment engine 113 may refine relevance rules of the ranking scheme while the system 100 is off-line and not actively retrieving patient diagnostic information and sorting the problem list. In another exemplary embodiment of step 207, in an on-line machine-learning method, the ranking method adjustment engine 113 may refine relevance rules of the ranking scheme in between the system 100 sessions of applying a ranking scheme to sort the problem list.

In another exemplary embodiment of step 207, the server for system 100 collects ranking data on user selections for the advanced problem list display on display 106, from different users, for system 100. In this exemplary embodiment, the ranking method adjustment engine 113 may refine the relevance values for the ranking scheme of the problem list ranking engine 111 by incorporating data on user ranking selections from the server.

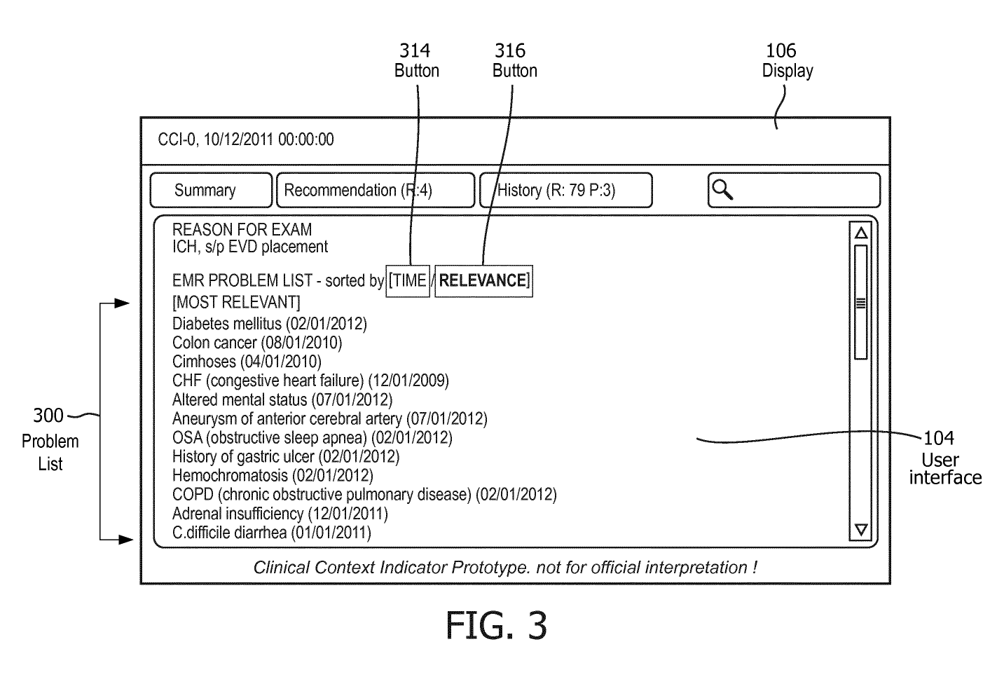

FIG. 3 shows an exemplary embodiment of ranking the problem list on a display 106 using user interface 104, which ranks the problem list of patient diagnostic information items according to relevance to the current imaging exam. FIG. 3 shows the diagnostic information items of problem list 300 ranked in order of decreasing relevance, with the most relevant items at the top of the problem list. In this exemplary embodiment, ranking scheme 312 (a relevance ranking scheme) is applied to sort the problem list. In this exemplary embodiment, the user may choose to apply ranking scheme 310 (a chronological ranking scheme) by clicking on the “TIME” button 314, or apply the ranking scheme 312 (a relevance ranking scheme) by clicking on the “RELEVANCE” button 316 on the user interface 104.

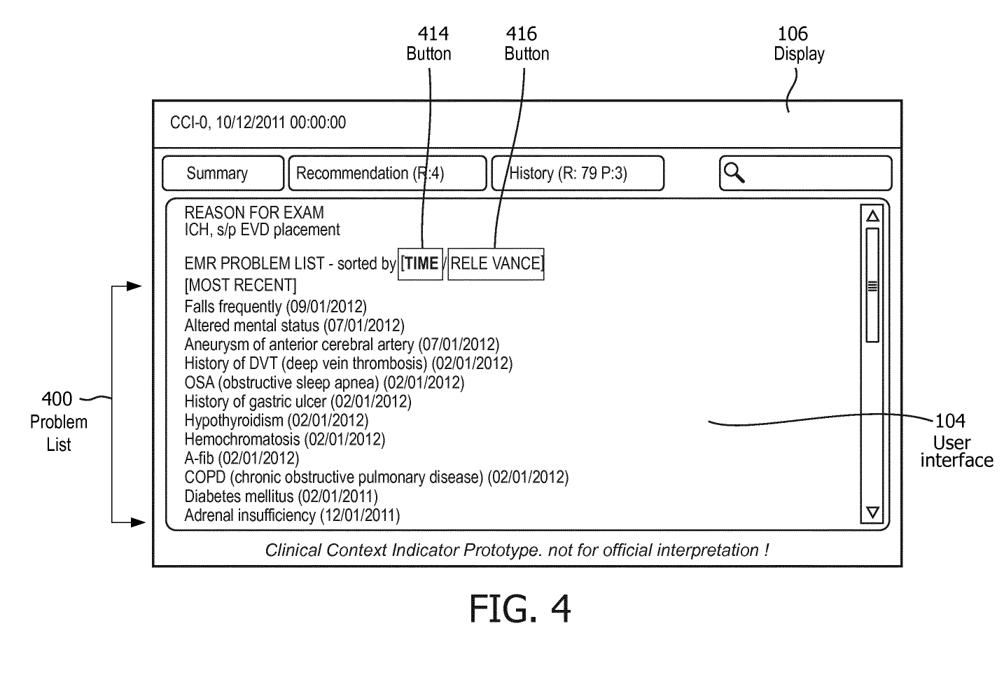

FIG. 4 shows an exemplary embodiment of ranking the problem list 400 on a display 106 using user interface 104, which ranks the problem list of patient diagnostic information items in reverse chronological order, with the most recent items at the top of the ranked problem list 400. In this exemplary embodiment, ranking scheme 410 (a chronological ranking scheme) is applied to sort the problem list. In this exemplary embodiment, the user may choose to apply ranking scheme 410 (a chronological ranking scheme) by clicking on the “TIME” button 414, or apply ranking scheme 412 (a relevance ranking scheme) by clicking on the “RELEVANCE” button 416 on the user interface 104.

Those skilled in the art will understand that the above-described exemplary embodiments may be implemented in any number of manners, including, as a separate software module, as a combination of hardware and software, etc. For example, the context construction engine 110, problem list ranking engine 111, medical document reporting engine 112, and ranking method adjustment engine 113 may be programs containing lines of code that, when compiled, may be executed on a processor.

It will be apparent to those skilled in the art that various modifications may be made to the disclosed exemplary embodiments and methods and alternatives without departing from the spirit or scope of the disclosure. Thus, it is intended that the present disclosure cover the modifications and variations provided that they come within the scope of the appended claims and their equivalents.

1. A method, comprising:
retrieving a problem list with active diagnostic information items for a patient;
constructing a clinical context for a current imaging exam based on retrieved relevant diagnostic information for the current imaging exam;
determining a ranking scheme with relevance rules based on the clinical context, wherein the relevance rules rank a relevance of the diagnostic information items of the problem list based on the clinical context for the current imaging exam, wherein determining the ranking scheme comprises modeling relevance according to one or more relevance themes, and at least one of:
mapping numerical relevance intervals to respective low, medium, and high relevance levels; or
mapping numerical relevance values to respective low, medium, and high relevance levels;
selecting a ranking scheme; and
implementing the selected ranking scheme to sort the diagnostic information items of the problem list.

2. The method of claim 1, further comprising:
displaying the sorted problem list; and
applying user selection of the diagnostic information items to the problem list.

3. The method of claim 1, wherein selecting a ranking scheme comprises:
selecting a chronological or relevance ranking scheme.

4. The method of claim 2, further comprising:
converting the user selection of the problem list to natural language statements; and
applying the user selection to refine the relevance rules of the determined ranking scheme.

5. The method of claim 3, further comprising:
sorting the problem list with the refined relevance rules of the determined ranking scheme.

6. The method of claim 1, wherein the relevant diagnostic information comprises at least one of: imaging exam modality, mode of image acquisition for the imaging exam, and anatomy imaged in the imaging exam.

7. (canceled)

8. The method of claim 7, wherein the relevance themes comprise: medical domain specialty, user profile, user, and exam date.

9. The method of claim 1, wherein determining the ranking scheme comprises:
creating relevance rules based on an International Classification of Diseases (ICD) hierarchy to map ICD codes to relevance values in a look-up table based on the clinical context parameters.

10. The method of claim 9, wherein creating the relevance rules based on an ICD hierarchy comprises at least one of:
setting an identical relevance value for an ICD node and its subordinate ICD codes; and
setting the identical relevance value for an ICD node and its subordinate ICD codes, in view of a date of entry for the ICD code.

11. The method of claim 2, wherein applying user selection of diagnostic information items to the problem list comprises one or more of:
selecting the diagnostic information items; or
removing pre-selected diagnostic information items, wherein each diagnostic information item comprises an ICD code.

12. The method of claim 4, wherein converting the user selection of the problem list to natural language statements comprises:
applying a template for converting the problem list to natural language statements; and
making the natural language statements accessible for user applications.

13. The method of claim 4, wherein refining the relevance rules of the determined ranking scheme comprises:
incorporating the user selection of diagnostic information for the problem list as relevance exception rules.

14. The method of claim 4, wherein refining the relevance rules of the determined ranking scheme comprises:
refining relevance values for the determined ranking scheme in a look-up table by requesting user feedback on parameters of the clinical context, for an International Classification of Diseases (ICD) hierarchy, wherein the look-up table maps ICD codes to the relevance values based on the parameters of the clinical context.

15. A system, comprising:
a non-transitory computer readable storage medium storing an executable program; and
a processor executing the executable program to cause the processor to:
retrieve a problem list with active diagnostic information items for a patient;
construct a clinical context for a current imaging exam based on retrieved relevant diagnostic information for the current imaging exam;
determine a ranking scheme with relevance rules based on the clinical context, wherein the relevance rules rank a relevance of problem list diagnostic information items based on the clinical context for the current imaging exam, wherein determining the ranking scheme comprises modeling relevance according to one or more relevance themes, and at least one of:
mapping numerical relevance value intervals to respective low, medium, and high relevance levels; or
mapping numerical relevance values to respective low, medium, and high relevance levels.
select a ranking scheme; and
implement the selected ranking scheme to sort the problem list diagnostic information items.

16. The system of claim 14, wherein the processor executes the executable program to cause the processor to:
display the sorted problem list; and
apply user selection of the diagnostic information items to the problem list.

17. The system of claim 16, wherein the processor executes the executable program to cause the processor to:
convert the user selections of the problem list to natural language statements; and
apply the user selection to refine the relevance rules of the ranking scheme.

18. The method of claim 15, wherein determining the ranking scheme comprises:
modeling relevance according to one or more relevance themes of medical domain specialty, user profile, user, and exam date; and at least one of:
mapping numerical relevance value intervals to respective low, medium, and high relevance levels; or
mapping numerical relevance values to respective low, medium, and high relevance levels.

19. The method of claim 15, wherein refining the relevance rules of the determined ranking scheme comprises:
refining relevance values for the determined ranking scheme in a look-up table by requesting user feedback on parameters of the clinical context, for an International Classification of Diseases (ICD) hierarchy, wherein the look-up table maps ICD codes to the relevance values based on the parameters of the clinical context.

20. A non-transitory computer-readable storage medium including a set of instructions executable by a processor, the set of instructions, when executed by the processor, causing the processor to perform operations, comprising:
retrieving a problem list with active diagnostic information items for a patient;
constructing a clinical context for a current imaging exam based on retrieved relevant diagnostic information for the current imaging exam;
determining a ranking scheme with relevance rules based on the clinical context, wherein the relevance rules rank a relevance of problem list diagnostic information items based on the clinical context for the current imaging exam, wherein determining the ranking scheme comprises modeling relevance according to one or more relevance themes, and at least one of:
mapping numerical relevance value intervals to respective low, medium, and high relevance levels; or
mapping numerical relevance values to respective low, medium, and high relevance levels.
selecting a ranking scheme; and
implementing the selected ranking scheme to sort the problem list diagnostic information items.
